(19) United States
(12) Patent Application Publication
(10) Pub. No.: US 2015/0230774 A1
(43) Pub. Date: Aug. 20, 2015

(54) BLOOD PRESSURE MONITOR AND METHOD

(71) Applicant: Le Thai, Phoenix, AZ (US)

(72) Inventor: Le Thai, Phoenix, AZ (US)

(21) Appl. No.: 14/704,805

(22) Filed: May 5, 2015

Related U.S. Application Data
(63) Continuation-in-part of application No. PCT/US2013/069275, filed on Nov. 8, 2013.
(60) Provisional application No. 61/723,910, filed on Nov. 8, 2012.

(51) Int. Cl. A61B 8/04; A61B 8/06; A61B 8/08; A61B 5/022

(52) U.S. Cl. CPC A61B 8/04; A61B 5/02225; A61B 8/06; A61B 8/488

(57) ABSTRACT

A method is provided for noninvasive, continuous, real-time monitoring of a patient's arterial blood pressure using Doppler probes and a blood pressure cuff for measurement of a patient's systolic and diastolic blood pressures at a major distal artery. A continuous Doppler blood flow velocity measurement is used to generate a waveform signal correlating to a cuff's measurements of systolic and diastolic pressures. In addition, a method determines cerebral blood perfusion by providing at least two Doppler probes, one of which is placed at a distal artery with a blood pressure cuff and another of which is placed over the carotid artery or the middle cerebral artery. An algorithm calculates systolic and diastolic pressures at the distal artery and at the carotid artery or the middle cerebral artery as a function of the continuously measured Doppler blood flow velocities.

This application is a continuation-in-part of pending International Patent Application PCT/US2013/069275 filed Nov. 8, 2013, that claims the benefit of expired U.S. Provisional Patent Application No. 61/723,910, filed Nov. 8, 2012.

### TECHNICAL FIELD

The present invention relates to continuous, noninvasive blood pressure monitoring using a combination of a blood pressure cuff and a Doppler ultrasound probe or probes.

### BACKGROUND

The predominant method for clinical measurement of blood pressure is the noninvasive auscultatory method using a stethoscope and a sphygmomanometer cuff. The medical practitioner listens with the stethoscope at the brachial artery while slowly releasing the pressure in the cuff. The systolic pressure is the pressure at which the first “whooshing” sound of blood flowing in the artery is heard. The diastolic pressure is the pressure at which no sound is heard.

Another noninvasive method for determining blood pressure is the use of a sphygmomanometer cuff with an electronic transducer to measure oscillations (oscillometer). An algorithm is used to compute the values of the systolic and diastolic pressures. This method is considered less accurate than the auscultatory method, although it is simpler to use. However, a continuous measurement of blood pressure cannot be obtained using either this method or the auscultatory method.

Noninvasive methods used to determine continuous arterial blood pressure by incorporating an inflatable finger cuff with a photo electric plethysmograph are commercially available from Finapres, Nexfin and CNAP. The principle applied in these devices is to balance equal pressures on either side of the wall of an artery by clamping the artery to a certain volume. Arterial pressure from the finger cuff pressure data can be used to continuously calculate the systolic and diastolic pressures.

Continuous blood pressure monitoring can be achieved by invasive techniques such as arterial lines, which require insertion of catheters into arteries with concomitant risks such as thrombosis, thromboembolism, infection, hematoma, and air emboli. Given these risks, arterial lines are not used for routine blood pressure monitoring.

Blood pressure monitoring is very important in surgery and emergency situations. It has been estimated that there are 400,000 operating rooms in the world. Furthermore, there are a very large number of intensive care unit beds whose occupants require monitoring. Other prime locations include radiology suites, dialysis units and specialty floor units.

### BRIEF SUMMARY OF THE INVENTION

In one embodiment, there is provided a method for noninvasive, continuous, real time monitoring of a patient's arterial blood pressure. The method has the steps of a) providing a blood pressure cuff and placing the cuff around a limb of the patient; b) providing a Doppler ultrasound probe, positioning the probe over a distal artery below the cuff, and continuously measuring Doppler blood flow velocities with the probe; c) inputting the Doppler blood flow velocities into a processor, wherein the processor generates a waveform signal of the Doppler blood flow velocities, d) inflating the cuff and measuring diastolic blood pressure at a cuff pressure at which a sustained change in Doppler blood flow velocity occurs; e) inflating the cuff further and measuring systolic blood pressure at a cuff pressure at which Doppler blood flow velocity is zero; f) deflating the cuff; g) correlating the Doppler waveform signal peak of maximum blood flow velocity to the systolic blood pressure and the Doppler waveform signal trough of end diastolic minimum velocity to the diastolic blood pressure; and h) generating calculated systolic and diastolic pressures with an algorithm as a function of the continuously measured Doppler blood flow velocities.

Optionally, the method also includes repetition of steps d) to g) are repeated at an interval of time to recalibrate the Doppler blood flow velocities to the systolic and diastolic blood pressures. Optionally, recalibration can be timed for selected intervals, such as about 3, 4, 5, 6, 7, 8, 9, or 10 minutes. Alternatively, the cuff pressure is measured by a sphygmomanometer. In addition the method measures mean arterial blood pressure. In a variation on step f, there is measuring systolic blood pressure at a cuff pressure at which the Doppler probe indicates an initial blood flow velocity, and measuring diastolic blood pressure at a cuff pressure at which the Doppler probe signal becomes muffled. Optionally, the method further calls for generating continuous measurements of systolic and diastolic pressures by continuously repeating steps d) to f), wherein deflating the cuff in step f) is stopped at measurement of diastolic blood pressure and followed by inflating the cuff in repeated step d). Optionally, the Doppler probe is positioned over a major artery.

In yet another embodiment, there is a method for noninvasive, continuous, real time monitoring of arterial blood pressure at the carotid artery of a patient. Here, the steps include a) providing a Doppler ultrasound probe and blood pressure cuff and placing the cuff around a limb of the patient over a distal artery; b) providing a second Doppler ultrasound probe, positioning the probe over a carotid artery in the neck, and continuously measuring Doppler blood flow velocities with the probes; c) inputting the Doppler blood flow velocities into a processor, wherein the processor generates a waveform signal of the Doppler blood flow velocities; d) measuring the vertical height difference between the cuff and the carotid artery; e) inflating the cuff and measuring diastolic blood pressure at a cuff pressure at which a sustained change in Doppler blood flow velocity occurs; f) inflating the cuff further and measuring systolic blood pressure at a cuff pressure at which Doppler blood flow velocity is zero; g) deflating the cuff; h) determining a corrected diastolic and systolic blood pressure at the carotid artery as a function of the height difference, wherein 1 centimeter of height is equal to a drop of 0.77 mmHg in pressure; i) correlating the Doppler waveform signal peak of maximum blood flow velocity to the corrected systolic blood pressure and the Doppler waveform signal trough of end diastolic minimum velocity to the corrected diastolic blood pressure; and j) generating calculated systolic and diastolic pressures with an algorithm as a function of the continuously measured Doppler blood flow velocities.

In yet another embodiment, there is provided a system for noninvasive, continuous, real time monitoring of arterial blood pressure of a patient. The system includes a blood pressure cuff; at least one Doppler ultrasound probe; a processor for generating a waveform signal of Doppler blood flow velocities; a processor for correlating the waveform signal to blood pressures determined with the blood pressure cuff; and a processor for generating systolic and diastolic blood pressures with an algorithm as a function of the Doppler blood flow velocities.

### DETAILED DESCRIPTION

As an anesthesiologist, I have found significant problems with monitoring blood pressure in critical situations. One available method has an inflatable finger cuff that is intended to provide data on blood pressure; however, it has been reported that signals maybe hard to detect in some patients, particularly in critically ill or pediatric patients. The data are not as dependable as invasive blood pressure data.

The use of an inflatable finger cuff with a photo electric plethysmograph enables continuous arterial blood pressure monitoring by generating a waveform that is correlated to blood pressures. However, fingertip measurements often fail because patients turn cold during surgery and respond with peripheral vasoconstriction (and low pressure). Furthermore, these systems are complicated and do not work well under low pressure conditions, such as shock, since they rely on finger blood flow which is compromised in low pressure states.

A different model wraps around two fingers, but it too has failed in critically ill patients, and its data are not equivalent to invasive blood pressure data. This method of measuring blood pressure uses continuous, noninvasive monitoring with a combination of a blood pressure cuff and photo electric plethysmographs. Yet another method operates off a T-line on the patient's wrist using applanation tonometry. The T-line can be difficult to use, is sensitive to patient motion, and is prone to artifacts. The output data are not equivalent to invasive blood pressure data.

The preferred invasive blood pressure data require the placement of an arterial line, to which is attached a non-compressible line filled with saline. The saline line is in communication with the pressure transducer and an automatic flushing system with a pressure bag. Another blood pressure method uses an oscillometric cuff equipped with a piezoelectric pressure sensor wrapped around the upper arm, where blood vessels are less prone to temperature-related changes that constrict distal vessels in the fingers. The air tubes to the cuff attach to a device with a pump for maintaining pressure in the cuff and a minicomputer to convert the mean pressure to systolic and diastolic pressures. Inaccuracies have ranged from about 3% to over 7%.

The predominant blood pressure monitoring method utilizing a sphygmomanometer has not allowed for continuous blood pressure monitoring. For example, a standard oscillometric blood pressure cuff in the operating room limits sampling of the patient's pressure to every 3-5 minutes. However, much can and does happen in the 3-5 minutes interval between blood pressure measurements. Thus, I have observed a need for continuous, noninvasive monitoring of blood pressure.

The “gold standard” for measuring low flow, low pressure blood flow is Doppler ultrasound including an ultrasound device and a sphygmomanometer. This procedure is typically performed at the upper arm where it records systolic pressure in the brachial artery or in the lower leg near the ankle

Options for monitoring cerebral perfusion are even more limited. On occasion, a transcranial Doppler test is run, but it requires an ultrasound technician, who later reports the results, a system ill-suited to continuous non-invasive monitoring. Cerebral oximetry during coronary bypass surgery offered no difference in overall incidence of adverse complications but significantly more major organ morbidity and mortality without it. However, cerebral oximetry results did not correlate with the “gold standard” of EEG and SSEP. Numerous studies have reported false positives and false negatives for current brain perfusion assessment techniques. Yet another way of assessing brain function is use of a Doppler sonography transducer positioned at the neck portion of the carotid artery or at the anterior temple or the middle cerebral artery (transcranial Doppler).

I have an improved way to monitor transcranial pressure, particularly blood pressure in the middle cerebral artery in the brain. Two monitors are placed on the body. The first is a transcranial Doppler probe at the anterior temple (over the middle cerebral artery). The second is a Doppler probe over a distal artery. Blood flow velocity data from the Doppler probe over the middle cerebral artery is converted into blood pressure using the calibration and algorithm generated by the distal Doppler probe and cuff system. Alternatively, the cuff pressure is measured by an oscillometric blood pressure cuff positioned on the same or contralateral arm as the Doppler probe. The measured systolic and diastolic pressures are correlated with the peak and trough blood flow velocities.

The methods incorporate measurements that are made by determining blood flow velocities as a simple function of Doppler ultrasound probe measurement. Other, more complicated methods utilizing Doppler measurement have been described. For example, U.S. Pat. No. 5,241,964 describes blood pressure determinations with a Doppler probe that measures the arterial resonant frequency of the blood vessel, employing the artery as the pressure transducer. Published PCT Application WO 2010048528 A2 describes blood pressure measurement using a Doppler probe to measure the cross sectional area of the artery, the blood vessel's compliance, and employs the blood vessel as a pressure transducer. This method may yield inaccurate results due to, for example, vasodilation, vasoconstriction of the patient, motion of the patient, and motion due to manipulation of the patient during surgery.

My methods advantageously provide noninvasive, continuous real time monitoring of blood pressure by converting blood flow velocities from Doppler probe measurement made over a major artery, rather than a distant peripheral site, such as a finger. The blood flow velocities are made as a simple function of Doppler measurements, which are calibrated to a sphygmomanometer or oscillometric blood pressure cuff. This calibration does not include complications from Doppler measurement that involve measuring factors such as arterial resonant frequency, arterial cross sectional area, blood vessel compliance, and use of the blood vessel as a pressure transducer. My methods also allow the noninvasive, continuous monitoring of the systolic and diastolic pressures at the carotid artery (as an estimate of the cerebral perfusion pressure) and at the middle cerebral artery, thereby alleviating the risks of cerebral hypoperfusion and ischemic injury in at risk patients.

My method allows the measurement of the velocity time integral that is useful in calculating cardiac output. The heart rate can also be determined by the number of waves per minute and monitored as a Doppler based heart rate monitor, with or without a blood pressure cuff (e.g., Doppler “wristwatch”).

Reference throughout this specification to an “embodiment,” an “example” or similar language means that a particular feature, structure, characteristic, or combinations thereof described in connection with the embodiment is included in at least one embodiment of the present invention. Thus appearances of the phrases an “embodiment,” and “example,” and similar language throughout this specification may, but do not necessarily, all refer to the same embodiment, to different embodiments, or to one or more of the figures. Additionally, reference to the words “embodiment,” “example” or the like for two or more features, elements, etc., does not mean that the features are necessarily related, dissimilar, the same, etc.

Each statement of an embodiment or example is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional un-recited elements or method steps. “Comprising” is to be interpreted broadly and including the more restrictive terms “consisting of” and “consisting essentially of.”

Reference throughout this specification to features, advantages, or similar language does not imply that all of features and advantages that may be realized with the present invention should be or are in any single embodiment of the invention. Rather, language referring to the features and advantages is understood to mean that a specific feature, advantage or characteristic described in connection with an embodiment is included in at least one embodiment of the present invention. Thus, discussion of the features and advantages, and similar language, throughout this specification may, but does not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description or may be learned by the practice of the invention as set forth.

My system and methods provide noninvasive, continuous, real time monitoring of arterial blood pressure. The methods utilize a combination of a blood pressure cuff and a Doppler ultrasound probe. In particular, the methods entail measuring blood flow velocities at a major artery, including but not limited to the middle cerebral, carotid, brachial, radial, and dorsalis pedis, with a Doppler probe, generating a waveform signal as a function of the velocities, and calibrating or correlating the waveform signal to cuff measurements of systolic and diastolic pressures. An algorithm generates calculated systolic and diastolic pressures at the major artery as a function of the continuously measured Doppler blood flow velocities.

In one embodiment, a method of noninvasive, real time monitoring of arterial blood pressure is carried out as follows. A sphygmomanometer cuff (or alternatively, an oscillometric cuff) is connected to a limb of a patient, such as the upper arm or other convenient sites, such as the lower arm or lower leg. A cuff of the correct size is wrapped around the limb. A first Doppler ultrasound probe is placed over a major artery that preferably is distal to and below the cuff. The major, distal artery is selected based on the particular cuff location. For example, the brachial artery is used for an upper arm cuff, the radial artery for lower arm cuff, and the dorsalis pedis artery for a lower leg cuff. The Doppler ultrasound probe can be separate from the cuff, or for ease of use, can be incorporated into the blood pressure cuff such that the ultrasound probe is attached to the cuff. Care is taken to ensure that the Doppler probe is positioned over the artery. Blood flow velocities are continuously measured by the Doppler ultrasound probe and the data entered electronically into a monitor, which contains a processor that generates a waveform signal.

The Doppler blood flow velocities and corresponding waveform signal are calibrated (correlated) to blood pressure measurements made with the blood pressure cuff. To measure blood pressure, the cuff is slowly and continuously inflated. The diastolic blood pressure is the cuff pressure at which there is a sustained change in Doppler blood flow velocity, which corresponds with the end diastolic minimum velocity. The cuff continues to be inflated. The systolic blood pressure is the cuff pressure at which the Doppler blood flow velocity becomes zero, that is, the blood flow stops. The cuff is then deflated. The systolic and diastolic pressures can also be measured as the cuff is gradually deflated. The systolic blood pressure is the cuff pressure at which the Doppler signal indicates an initial blood flow velocity. The diastolic blood pressure is the cuff pressure at which the Doppler signal becomes muffled, corresponding to the end diastolic minimum velocity. The systolic, diastolic and mean arterial blood pressures can also be measured using the oscillometer function of the cuff.

The waveform signal of the Doppler blood flow velocities is calibrated to the blood pressure by a processor in the system monitor. The waveform signal of the Doppler blood flow velocities correlates the maximum blood flow velocity (peak of the wave) to the systolic blood pressure and the near zero blood flow velocity (trough, end diastolic minimum velocity) to the diastolic blood pressure. An algorithm is used to generate calculated systolic and diastolic pressures as a function of the continuously measured Doppler blood flow velocities. An example of the derivation of such an algorithm conversion method is found in Elter et al., Noninvasive and non-occlusive determination of blood pressure using laser Doppler flowmetry. This reference is available online at: http://proceedings.spiedigitallibrary.org/proceeding.aspx?articleid=976274. The formal citation for this reference is Proc. SPIE 3596, Specialty Fiber Optics for Medical Applications, 188 (Apr. 21, 1999). This reference explains a non-occlusive (without a blood pressure cuff, contrary to the present invention) method for calculating blood pressure from measured blood flow velocities, graphing the two and otherwise obtaining the necessary parameters and constants for a given set of data. Elter et al. used Navier Stokes differential equations and simulations with a laser Doppler flow sensor at the radialis artery.

That is, by an algorithm correlating the change in Doppler blood flow velocities to the change in pressure by analyzing the Doppler waveform signal. Continuous systolic, diastolic and mean arterial pressures are displayed on the monitor, along with the continuous display of an arterial blood pressure waveform and the corresponding systolic and diastolic pressures.

The system is recalibrated at certain intervals with cuff measured systolic and diastolic arterial pressures using the above steps. The rest interval between recalibration allows for the perfusion of the limb and enhances patient comfort. In a routine mode for awake patients, the recalibration of the monitor is performed about every 3 to 5, 6 to 8, or up to every 9 to 10 minutes for patient comfort. In an anesthesia mode for anesthetized patients, the recalibration of the monitor is performed about every 3 minutes. In the case of an emergency, such as a sudden drop in blood flow velocity, a systolic blood pressure lower than a preset amount, or other emergency situations or conditions, an emergency mode is used. In this mode, the blood pressure cuff is programmed to continuously hover between the peak and trough Doppler blood flow velocities, generating continuous direct (not algorithm generated) measurements of systolic and diastolic pressures. This mode can be sustained for a significant amount of time without compromising the perfusion to the limb. It is preferred that the time not exceed one hour unless essential. Shorter times (such as 30, 40, 45, 50 and 55 minutes) are preferred.

In another embodiment, the methods disclosed herein may also be used for noninvasive, continuous, real time monitoring of arterial blood pressure at the carotid artery and/or the middle cerebral artery as an estimate of the cerebral perfusion pressure, alleviating the risks of cerebral hypoperfusion and ischemic injury in at risk patients. The monitoring of the blood pressure at the carotid artery and the middle cerebral artery may be performed independently, or in conjunction with the monitoring at another major artery located distal to the blood pressure cuff. In this scenario of two probes, a first Doppler ultrasound probe is used at the major, distal artery, and a second Doppler ultrasound probe is used at the carotid artery or the middle cerebral artery.

In an embodiment of monitoring of arterial blood pressure at the carotid artery or the middle cerebral artery, a Doppler ultrasound probe is positioned over the carotid artery in the neck (either right or left side) or over the middle cerebral artery (Transcranial Doppler) and a second Doppler ultrasound probe is positioned below a blood pressure cuff over a major distal artery. Blood flow velocities are continuously measured by the Doppler ultrasound probes and the data entered electronically into a monitor, in which a processor generates a waveform signal. The vertical height difference between the cuff and the carotid artery or the middle cerebral artery is determined. The blood pressure at the carotid artery in the neck or the middle cerebral artery is the blood pressure measured at the cuff corrected for the height difference (1 centimeter of height is equal to a drop of 0.77 mm Hg in pressure). Preferably, the height difference is determined by a measuring tape system incorporated in the cuff, whereby the length of the segment of the measuring tape pulled determines the height difference. More preferably, the measuring tape system information is automatically entered into the blood pressure monitor or monitoring system. The height difference is accounted for by the monitor, whether input manually or automatically, to automatically correct the systolic and diastolic blood pressures generated by the cuff/Doppler probe placed over the major, distal artery.

The automatically corrected blood pressure data are correlated with the Doppler waveform signal at the carotid artery or the middle cerebral artery to generate a continuous real time arterial blood pressure tracing at the carotid artery or the middle cerebral artery. In a preferred embodiment, the calculated blood pressures at the carotid artery or the middle cerebral artery, the generated arterial waveform signal, and an auditory sonogram are displayed on the system monitor for a continuous monitoring of cerebral perfusion. This continuous monitoring is crucial when the patient is in a “sitting up” position, which is accompanied by increased risks of cerebral hypoperfusion and ischemic injury.

The methods disclosed herein of noninvasive, continuous measurement of arterial blood pressure are carried out with a blood pressure monitoring system. The components of the system include, but are not limited to, a blood pressure cuff, a Doppler ultrasound probe or probes, a processor for generating a waveform signal of the Doppler blood flow velocities, a processor for correlating the waveform signal to blood pressures determined with the blood pressure cuff, and a processor for generating systolic and diastolic blood pressures with an algorithm as a function of the Doppler blood flow velocities.

The system will contain at least one Doppler ultrasound probe, and will optionally contain a second probe. A single Doppler ultrasound probe may be used to measure arterial blood pressure at a major artery distal to the blood pressure cuff and derive carotid or middle cerebral arterial blood pressure by correcting for height difference, while two probes may be used to measure both distal arterial blood pressure and carotid or middle cerebral arterial blood pressure. The Doppler probe(s) may be separate from the blood pressure cuff or, alternatively, integrated with the blood pressure cuff for ease of use. To facilitate corrected measurement of carotid arterial blood pressure, the cuff may include a measuring tape for measurement of the height difference between the carotid or the middle cerebral artery and the cuff.

The processors are contained within a monitor for the system, which includes displays for an arterial blood pressure waveform and the corresponding systolic and diastolic pressures. The monitor additionally may display mean arterial blood pressure and an auditory arterial sonogram. In the embodiment of monitoring carotid artery pressure, the monitor may also display an auditory carotid sonogram. The system monitor preferably will also include componentry or control componentry for operating the system, including inflation and deflation of the cuff, recording pressures from the cuff, as well as receiving and processing information from the Doppler probe(s).

Although specific embodiments have been illustrated and described herein, those of ordinary skill in the art will appreciate that any arrangement calculated to achieve same purposes can be substituted for the specific embodiments shown. This disclosure is intended to cover any and all adaptations or variations of various embodiments of the invention. It is to be understood that the above description has been made in an illustrative fashion, and not a restrictive one. Combinations of the above embodiments, and other embodiments not specifically described herein will be apparent to those of skill in the art upon reviewing the above description. The scope of various embodiments of the invention includes any other applications in which the above structures and methods are used. Therefore, the scope of various embodiments of the invention should be determined with reference to the appended claims, along with the full range of equivalents to which such claims are entitled.

In the foregoing description, if various features are grouped together in a single embodiment for the purpose of streamlining the disclosure, this method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims, and such other claims as may later be added, are hereby incorporated into the description of the embodiments of the invention, with each claim standing on its own as a separate preferred embodiment.

1. A method for noninvasive, continuous, real time monitoring of arterial blood pressure of a patient, comprising:
a) providing a blood pressure cuff and placing the cuff around a limb of the patient;
b) providing a Doppler ultrasound probe, positioning the probe over a distal artery below the cuff, and continuously measuring Doppler blood flow velocities with the probe;
c) inputting the Doppler blood flow velocities into a processor, wherein the processor generates a waveform signal of the Doppler blood flow velocities;
d) inflating the cuff and measuring diastolic blood pressure at a cuff pressure at which a sustained change in Doppler blood flow velocity occurs;
e) inflating the cuff further and measuring systolic blood pressure at a cuff pressure at which Doppler blood flow velocity is zero;
f) deflating the cuff;
g) correlating the Doppler waveform signal peak of maximum blood flow velocity to the systolic blood pressure and the Doppler waveform signal trough of end diastolic minimum velocity to the diastolic blood pressure; and
h) generating calculated systolic and diastolic pressures with an algorithm as a function of the continuously measured Doppler blood flow velocities.

2. The method of claim 1, wherein steps d) to g) are repeated at an interval of time to recalibrate the Doppler blood flow velocities to the systolic and diastolic blood pressures.

3. The method of claim 2, wherein the interval of time is about 3, 4, 5, 6, 7, 8, 9, or 10 minutes.

4. The method of claim 1, wherein the cuff pressure is measured by a sphygmomanometer.

5. The method of claim 1, wherein the cuff pressure is measured by an oscillometer.

6. The method of claim 5, further comprising measurement of mean arterial blood pressure.

7. The method of claim 1, step f) further comprising measuring systolic blood pressure at a cuff pressure at which the Doppler probe indicates an initial blood flow velocity, and measuring diastolic blood pressure at a cuff pressure at which the Doppler probe signal becomes muffled.

8. The method of claim 7, further comprising generating continuous measurements of systolic and diastolic pressures by continuously repeating steps d) to f), wherein deflating the cuff in step f) is stopped at measurement of diastolic blood pressure and followed by inflating the cuff in repeated step d).

9. The method of claim 1, wherein the Doppler probe is positioned over a major artery.

10. A method for noninvasive, continuous, real time monitoring of cranial perfusion via determining arterial blood pressure at the carotid artery of a patient, comprising:
a) providing a Doppler ultrasound probe and a blood pressure cuff and placing the cuff around a limb of the patient and the probe over a distal artery;
b) providing a second Doppler ultrasound probe, positioning the probe over a carotid artery in the neck, and continuously measuring Doppler blood flow velocities with the probes;
c) inputting the Doppler blood flow velocities into a processor, wherein the processor generates a waveform signal of the Doppler blood flow velocities;
d) measuring the vertical height difference between the cuff and the carotid artery;
e) inflating the cuff and measuring diastolic blood pressure at a cuff pressure at which a sustained change in Doppler blood flow velocity occurs;
f) inflating the cuff further and measuring systolic blood pressure at a cuff pressure at which Doppler blood flow velocity is zero;
g) deflating the cuff;
h) determining a corrected diastolic and systolic blood pressure at the carotid artery as a function of the height difference, wherein 1 centimeter of height is equal to a drop of 0.77 mmHg in pressure;
i) correlating the Doppler waveform signal peak of maximum blood flow velocity to the corrected systolic blood pressure and the Doppler waveform signal trough of end diastolic minimum velocity to the corrected diastolic blood pressure; and
j) generating calculated systolic and diastolic pressures with an algorithm as a function of the continuously measured Doppler blood flow velocities.

11. The method of claim 10 wherein the second Doppler probe is placed over the middle cerebral artery.

12. A system for noninvasive, continuous, real-time monitoring of arterial blood pressure of a patient, comprising:
a blood pressure cuff;
at least one Doppler ultrasound probe;
a processor for generating a continuous waveform signal of Doppler blood flow velocities;
a processor for correlating the waveform signal to blood pressures determined with the blood pressure cuff; and
a processor for generating by algorithm continuous, real-time systolic and diastolic blood pressures as a function of the continuous Doppler blood flow velocities.
